(19) United States
(12) Patent Application Publication
(10) Pub. No.: US 2007/0117781 A1
(43) Pub. Date: May 24, 2007

(54) Pharmaceutical formulation with improved stability

(75) Inventors: Amar Lulla, Maharashtra (IN); Geena Malhotra, Maharashtra (IN)

Correspondence Address: CONLEY ROSE, P.C., 5700 GRANITE PARKWAY, SUITE 330, PLANO, TX, 75024, US

(73) Assignee: Koninklijke Philips Electronics, N.V., Eindhoven (NL)

(21) Appl. No.: 10/574,135

(22) PCT Filed: Sep. 29, 2004
(86) PCT No.: PCT/GB04/04146

§ 371 Date: Oct. 18, 2006

(30) Foreign Application Priority Data
Sep. 29, 2003 (IN) 1023/MUM/2003

(51) Int. Cl. A61K 31/675; A61K 31/66

(52) U.S. Cl. 514/89; 514/102

(57) ABSTRACT

An oral formulation which includes an intragranular phase comprising a bisphosphonic acid derivative and at least one carbohydrate alcohol, together with an aqueous binder. A process of preparing the same and a therapeutic method employing such a formulation in the treatment of various skeletal diseases, such as systemic bone diseases including osteoporosis, osteoarthritis, Paget's disease, osteomalacia, multiple myeloma, and other forms of cancer, steroid therapy wherein the skeletal system is effected and age-related loss of bone mass, local disorders such as bone fractures and other such related disorders.

The present invention relates to an improved oral formulation for bisphosphonic acid derivatives, a process of preparing the same, therapeutic uses thereof and methods of treatment employing the same.

Bisphosphonic acid derivatives are very well known in the pharmaceutical field for use in the treatment of skeletal disorders. These bisphosphonic acids include, but are not limited to, clodronic acid, pamidronic acid, alendronic acid, risedronic acid, etidronic acid, ibandronic acid, tiludronic acid and other such therapeutic agents belonging to this class of compounds, and their salts and solvates. Bisphosphonic acid derivatives are active in calcium and phosphate metabolism mediated disorders.

Alendronate sodium, that is the monosodium salt of (4-amino-1-hydroxybutylidene) bisphosphonic acid, is taught by DE 3,016,289.

U.S. Pat. No. 6,554,967 describes a liquid formulation comprising alendronate monosodium trihydrate, together with sodium propyl paraben, sodium butyl paraben, sodium citrate dihydrate, citric acid anhydrous, sodium hydroxide to adjust pH and water as a vehicle.

WO 95/29679 relates to a process and a composition comprising bisphosphonic acid derivatives. The composition is made by the wet granulation techniques using water. The diluents comprise lactose and microcrystalline cellulose, which are wet granulated with the bisphosphonic acid derivative. However, a problem associated with the wet granulation of bisphosphonic acid derivatives with lactose as disclosed in WO 95/29679, is the potential degradation that can occur further to interaction of lactose present in the dosage form with a primary or a secondary amine group of the bisphosphonic acid derivatives.

Alendronic acid as the monosodium salt trihydrate is an active ingredient of the pharmaceutical oral dosage formulation available under the trade mark Fosamax, indicated for the treatment and prevention of osteoporosis. In addition to the active ingredient, this formulation further comprises microcrystalline cellulose, anhydrous lactose, croscarmellose sodium and magnesium stearate as excipients. Lactose, which is used in the Fosamax formulation in its anhydrous form, is generally used as a filler for solid dosage forms due to its excellent compressibility, high purity and stability.

The above degradation problems associated with bisphosphonic acid derivatives when present in lactose containing dosage forms is discussed in WO 01/85176. More specifically, it is described in WO 01/85176 that lactose may generate formulation incompatibilities with primary or secondary amine group containing compounds. It is further described that the incompatibilities are caused by the reaction between the reducing aldehyde moiety of lactose and the amine group present in the active ingredient. This reaction is known as the Maillard reaction.

The resulting degradation products are inactive. The formation of the degradation products is evidenced by a brown colouring of the final drug dosage form. The presence of water enhances the degradation [Handbook of Pharmaceutical Excipients, 2^{nd} edition, 1994, pg. 257 (ISBN 091730 60 8)].

The problem of browning of lactose containing dosage forms including alendronic acid and other bisphosphonic acid derivatives with a primary or a secondary amine group is also described in WO 94/12200. WO 94/12200 proposes a method of avoiding the interaction of lactose with bisphosphonic acid derivatives comprising an amine group in the molecule by providing a dry composition of the active ingredient and lactose. The process of preparation thereof comprises the direct blending of the dry mix without granulation or addition of water before compression.

U.S. Pat. No. 5,358,941, U.S. Pat. No. 6,090,410 and U.S. Pat. No. 5,681,590 also describe a dry mix for bisphosphonic acids along with lactose, in which the lactose used is essentially anhydrous. The process of preparation of the compositions involves direct compression of the dry mix.

U.S. Pat. No. 5,849,726 and U.S. Pat. No. 6,008,207 also describe a formulation of an anhydrous bisphosphonic acid derivative, namely anhydrous alendronate monosodium, together with anhydrous lactose and microcrystalline cellulose. The method of preparation as described therein is again direct compression of a dry mix formulation comprising the active ingredient, lactose and other ingredients such as microcrystalline cellulose, magnesium stearate and croscarmellose sodium.

Although WO 01/85176 acknowledges the prior art teaching as attempting to stabilize the bisphosphonic acid derivative formulations, WO 01/85176 further teaches that the prior art direct compression methods of preparing these formulations do not, however, solve the instability problems associated with bisphosphonic acid derivative formulations during long storage, especially in warm and damp conditions. WO 01/85176 thus describes a wet granulation method for preparation of a formulation comprising a bisphosphonic acid derivative along with a carbohydrate alcohol, such as D-mannitol. The described preparatory techniques, however, avoid direct contact of water with the bisphosphonic acid derivative and mannitol. More specifically, WO 01/85176 describes preparing a core of mannitol and cross-linked polyvinylpyrrolidone and polyvinylpyrrolidone by wet granulation and drying the resulting core to obtain granules. The granules are then combined with the active ingredient, lubricant and other excipients and the resulting blend is then compressed to form tablets. WO 01/85176 describes the quantity of mannitol as being in the range of 50 to 80%. It has been found, however, that such a high quantity of mannitol may create problems with compressibility and there thus remains a need for an improved formulation which overcomes both this problem and the problems of potential degradation associated with other prior art formulations. Despite describing a wet granulation process, WO 01/85176 clearly anticipates degradation when the bisphosphonic acid derivative, along with mannitol, is intimately mixed with water or any other such aqueous solvent.

It is thus an object of the present invention to provide an improved stable formulation for bisphosphonic acid derivatives, an improved process of preparing the same and methods of treatment employing such an improved formulation, in particular for the treatment or prevention of various skeletal diseases, including systemic bone diseases like osteoporosis, osteoarthritis, Paget's diseases, osteomalacia, multiple myeloma, and other forms of cancer, steroid therapy wherein the skeletal system is affected, age-related bone mass, local disorders, such as bone fractures and other such related disorders. In particular, it is an object of the present invention to provide an improved formulation with acceptable compression properties and also improved content uniformity of the drug, whilst avoiding the prior art degradation problems discussed above.

The present invention is thus concerned with an oral formulation comprising a bisphosphonic acid derivative, at least one carbohydrate alcohol and an aqueous binder, and more specifically there is now provided an oral formulation which includes an intragranular phase comprising a bisphosphonic acid derivative and at least one carbohydrate alcohol, together with an aqueous binder.

Surprisingly, it has thus been found by the present invention that a stable formulation comprising a bisphosphonic acid derivative can be prepared by intimately mixing the bisphosphonic acid derivative with a carbohydrate alcohol, whilst using an aqueous binder. A formulation so prepared by the simple techniques of the present invention has been found to be highly stable and does not result in degradation of the bisphosphonic acid derivative.

A further important advantage of the present invention associated with wet granulating the bisphosphonic acid derivative along with the carbohydrate alcohol using an aqueous binder is that it helps achieve a better content uniformity of the drug. When the bisphosphonic acid derivative is added as an extragranular ingredient as suggested in WO 01/85176, there is a possibility of the drug not being uniformly mixed and thus the present invention alleviates this problem by incorporating the drug intragranularly.

A particularly preferred feature of the present invention is that the formulations provided thereby do not contain lactose and as such the degradation problems of the prior art associated therewith are avoided by the present invention. Furthermore, in combination with the avoidance of degradation, the present invention achieves good content uniformity of the drug by incorporating the bisphosphonic acid derivative in the intragranular phase.

The active bisphosphonic acid derivative is preferably selected from the group consisting of 4-amino-1-hydroxybutylidene) bisphosphonic acid (alendronic acid), dichloromethylene bisphosphonic acid (clodronic acid), (1-hydroxy-3-(methylpentylamino)propylidene)bisphosphonic acid) (ibandronic acid), (1-hydroxyethylidene) diphosphonic acid (etidronic acid), (3-amino-1-hydroxypropylidene)bisphosphonic acid (pamidronic acid), [1-hydroxy-2-(3-pyridinyl)ethylidene]bisphosphonic acid (risedronic acid) and [[(4-chlorophenyl)thio]methylene]bisphosphonic acid (tiludronic acid), or a pharmaceutically acceptable derivative, salt, solvate, hydrate, prodrug, enantiomer or racemic mixture thereof, or any other compound of this class which is susceptible to degradation with lactose resulting in browning of the dosage form, including pharmaceutically acceptable salts, solvates, hydrates, prodrugs, enantiomers or racemic mixtures thereof. In a preferred embodiment, the active bisphosphonic acid derivative is present in a formulation according to the present invention in salt from, preferably as a sodium, disodium or trisodium salt, optionally in hydrated form, such as the monohydrate, dihydrate or trihydrate. Preferably, the bisphosphonic acid derivative is selected from the group consisting of alendronate sodium trihydrate, etidronate disodium and risedronate sodium monohydrate. In a preferred embodiment, the bisphosphonic acid derivative is alendronate sodium trihydrate. In an alternative preferred embodiment, the bisphosphonic acid derivative is etidronate disodium. In an alternative preferred embodiment, the bisphosphonic acid derivative is risedronate sodium monohydrate. The bisphosphonic acid derivative may suitably be present in the range of 0.5% to 40% with respect to the formulation.

A carbohydrate alcohol present in a formulation according to the present invention may be selected from the group consisting of mannitol, maltitol, sorbitol, lactitol, erythritol and xylitol, and also other such compounds of this class, including isomers and racemic mixtures thereof, and which preferably do not contain a reducing aldehyde moiety in their chemical structure. A preferred carbohydrate alcohol is mannitol. Suitably a carbohydrate alcohol is present in the formulation in the range of 15 to 90%, preferably 15 to 50% and more preferably in the range of 15 to 40%.

The intragranular phase may further comprise additional intragranular excipients, such as diluents and disintegrants.

The diluents may comprise one or more of starches and cellulose derivatives such as microcrystalline cellulose and powdered cellulose, calcium phosphate-dibasic, calcium sulfate, dextrates, dextrins, gums such as alginates, dextrose excipients and the like. The diluents may be present in the formulation in the range of 15 to 90%. The preferred diluent is microcrystalline cellulose.

The disintegrants suitably comprise one or more of low substituted hydroxypropyl cellulose, carboxymethyl cellulose, calcium carboxymethylcellulose, sodium carboxymethyl cellulose, sodium starch glycollate, crospovidone, croscarmellose sodium, starch, crystalline cellulose, hydroxypropyl starch, and partially pregelatinized starch. The disintegrant may be present in the formulation in the range of 5 to 20%. The preferred disintegrant is sodium starch glycollate.

The intragranular ingredients may be converted to granules by using suitable binders selected from natural and synthetic gums, celluloses such as hydroxypropyl cellulose, hydroxypropyl methylcellulose, carboxymethyl cellulose sodium, polyvinylpyrrolidones, starches, gelatins and povidones and other such pharmaceutically acceptable substances with cohesive properties. The binder may be present in the formulation in the range of 1 to 15%. The preferred binder is starch. The binder solution can be prepared using water.

The granules may be further lubricated by employing lubricants/glidants selected from the group consisting of talc, magnesium stearate, stearic acid, hydrogenated vegetable oils, glyceryl behenate, polyethylene glycols and their derivatives, sodium lauryl sulphate, sodium stearyl fumarate, and the like. The lubricants/glidants may be present in the formulation in the range of 0.5 to 5%. The preferred lubricant/glidant is magnesium stearate.

The present invention further provides a simple process of manufacturing a formulation as provided by the present invention substantially as herein described and which does not result in degradation of a bisphosphonic acid derivative present therein. According to the present invention, therefore, there is provided a process of preparing a formulation as described herein, which process comprises intimately mixing a bisphosphonic acid derivative and at least one carbohydrate alcohol to form a dry blend, wet granulating the dry blend with an aqueous binder so as to obtain an intragranular phase, and further formulating the resulting intragranular phase so as to provide a desired formulation according to the present invention. In this way in a process according to the present invention, the bisphosphonic acid derivative comes into direct contact with the aqueous binder. Suitably the process comprises forming a dry blend of the bisphosphonic acid derivative, at least one carbohydrate alcohol, together with other suitable intragranular excipients, and further formulating the granules of the intragranular phase suitably by compression to obtain a tablet, or encapsulating the granules to obtain capsules in accordance with the present invention.

A formulation as prepared in accordance with the present invention can be formulated as tablets, capsules, pellets, dry syrups, liquids and other suitable oral dosage forms. Preferably a formulation according to the present invention comprises a tablet or capsule.

A formulation as provided by the present invention can be used in the treatment of various skeletal diseases, such as systemic bone diseases including osteoporosis, osteoarthritis, Paget's disease, osteomalacia, multiple myeloma, and other forms of cancer, steroid therapy wherein the skeletal system is effected and age-related loss of bone mass, local disorders such as bone fractures and other such related disorders. According to the present invention, therefore, there is provided a method of treating or preventing a disease state which is ameliorated or eliminated by the administration of a bone resorption inhibitor, such as systemic bone diseases including osteoporosis, osteoarthritis, Paget's disease, osteomalacia, multiple myeloma, and other forms of cancer, steroid therapy wherein the skeletal system is effected and age-related loss of bone mass, local disorders such as bone fractures and other such related disorders, which method comprises administering an effective or prophylactic amount of a pharmaceutical formulation substantially as hereinbefore described.

A formulation in accordance with the present invention provides the active ingredient in a required therapeutic range. The formulations can be used to treat humans, particularly females who are post-menopausal, with an osteogenically effective amount of the bisphosphonic acid derivative to inhibit bone resorption. The term “bone resorption” as used herein, refers to treatment and prevention of bone loss, especially inhibiting the removal of existing bone either from the mineral phase and/or the organic matrix phase, through direct or indirect alteration of osteoclast formation or activity. Thus, the term “inhibitor of bone resorption” refers to agents that prevent bone loss by the direct or indirect alteration of osteoclast formation or activity and which may increase bone mass in the patient treatment populations. The term “osteogenically effective” as used herein denotes an amount that affects the turnover of mature bone. As used herein, an osteogenically effective dose is also pharmaceutically or therapeutically effective.

There is also provided by the present invention a process of reducing, or substantially eliminating, degradation products associated with a bisphosphonic acid derivative substantially as herein before described when present in a pharmaceutical formulation, especially a formulation including lactose, which process comprises formulating a bisphosphonic acid derivative together with at least one carbohydrate alcohol, in the presence of an aqueous binder, as an intragranular phase of an oral pharmaceutical formulation, and further formulating the thus formed intragranular phase so as to provide a pharmaceutical formulation according to the present invention. The present invention further provides use of an intragranular phase comprising a bisphosphonic acid derivative, together with at least one carbohydrate alcohol, prepared by wet granulation in the presence of an aqueous binder, in reducing or substantially eliminating degradation products associated with a bisphosphonic acid derivative present in a pharmaceutical formulation, especially a formulation including lactose.

It will be readily apparent to one skilled in the art that varying substitutions and modifications may be made to the invention disclosed herein without departing from the scope and spirit of the invention. Thus, it should be understood that although the present invention has been specifically disclosed by preferred embodiments and optional features, modification and variation of the concepts herein disclosed may be resorted to by those skilled in the art, and that such modifications and variations are considered to be falling within the scope of the invention.

The following examples are for the purpose of illustration of the invention only and are not intended in any way to limit the scope of the invention.

### EXAMPLE 1

| SR. | | QTY/TAB | QTY/TAB |
|---|---|---|---|
| NO. | INGREDIENTS | (mg) | (mg) |
| | INTRAGRANULAR INGREDIENTS | | |
| 1. | Alendronate Sodium Trihydrate | 35 | 70 |
| | equivalent to alendronic acid | | |
| 2. | Microcrystalline cellulose | 57.5 | 115.0 |
| 3. | Mannitol | 58.32 | 116.63 |
| 4. | Sodium Starch Glycollate | 7.5 | 15.0 |
| | BINDER SOLUTION | | |
| 5. | Starch | 1.5 | 3.0 |
| 6. | Purified Water | q.s. | q.s. |
| | EXTRAGRANULAR | | |
| | INGREDIENTS | | |
| 7. | Magnesium Stearate | 2.00 | 4.00 |
| 8. | Sodium Starch Glycollate | 2.5 | 5.00 |
| | TOTAL | 175.00 | 350.00 |

Process:

Pre-sifted alendronate sodium trihydrate, sodium starch glycollate, mannitol and microcrystalline cellulose were dry-mixed in the Fluidized Bed Processor for 5 minutes. Binder solution was prepared using starch and purified water. This binder solution was then sprayed over the dry mix at a specified rate and temperature to obtain granules. The granules were then spray dried and sifted. The granules were then blended in a suitable blender along with sodium starch glycollate and pre-sifted magnesium stearate. The granules were then compressed to form tablets.

### EXAMPLE 2

| SR. | | QTY/TAB |
|---|---|---|
| NO. | INGREDIENTS | (mg) |
| | INTRAGRANULAR INGREDIENTS | |
| 1. | Etidronate disodium equivalent to | 200 |
| | etidronic acid | |
| 2. | Microcrystalline cellulose | 158.20 |
| 3. | Mannitol | 156.64 |
| 4. | Sodium Starch Glycollate | 20.14 |
| | BINDER SOLUTION | |
| 5. | Starch | 4.0 |
| 6. | Purified Water | q.s. |
| | EXTRAGRANULAR | |
| | INGREDIENTS | |
| 7. | Magnesium Stearate | 5.52 |
| 8. | Sodium Starch Glycollate | 5.5 |
| | TOTAL | 550.00 |

Process:

Pre-sifted etidronate disodium, sodium starch glycollate, mannitol and microcrystalline cellulose were dry-mixed in the Fluidized Bed Equipment for 5 minutes. Binder solution was prepared using starch and purified water. This binder solution was then sprayed over the dry mix at a specified rate and temperature to obtain granules. The granules were then spray dried and sifted followed by mixing them in a suitable blender along with sodium starch glycollate and pre-sifted magnesium stearate. The granules were then filled in capsules.

### EXAMPLE 3

| SR. | | QTY/TAB |
|---|---|---|
| NO. | INGREDIENTS | (mg) |
| | INTRAGRANULAR INGREDIENTS | |
| 1. | Risedronate sodium monohydrate | 30 |
| | equivalent to Risedronic acid. | |
| 2. | Microcrystalline cellulose | 45.3 |
| 3. | Mannitol | 55.6 |
| 4. | Sodium Starch Glycollate | 4.9 |
| | BINDER SOLUTION | |
| 5. | Starch | 1 |
| 6. | Purified Water | q.s. |
| | EXTRAGRANULAR | |
| | INGREDIENTS | |
| 7. | Magnesium Stearate | 1.3 |
| 8. | Sodium Starch Glycollate | 1.6 |
| | TOTAL | 140.00 |

Process:

Pre-sifted risedronate sodium monohydrate, sodium starch glycollate, mannitol and microcrystalline cellulose were dry-mixed in the Fluidized Bed Equipment for 5 minutes. Binder solution was prepared using starch and purified water. This binder solution was then sprayed over the dry mix at a specified rate and temperature to obtain granules. The granules were then spray dried and sifted followed by mixing them in a suitable blender along with sodium starch glycollate and pre-sifted magnesium stearate. The granules were then filled in capsules.

1. An oral formulation which includes an intragranular phase comprising a bisphosphonic acid derivative and at least one carbohydrate alcohol, together with an aqueous binder.

2. A formulation according to claim 1, which does not contain lactose.

3. A formulation according to claim 1, wherein the bisphosphonic acid derivative is selected from the group consisting of alendronic acid, clodronic acid, ibandronic acid, etidronic acid, pamidronic acid, risedronic acid and tiludronic acid, or a pharmaceutically acceptable derivative, salt, solvate, hydrate, prodrug, enantiomer or racemic mixture thereof.

4. A formulation according to claim 1, wherein the bisphosphonic acid derivative is present in salt from.

5. A formulation according to claim 4, wherein the bisphosphonic acid derivative is present as a sodium, disodium or trisodium salt, optionally in hydrated form.

6. A formulation according to claim 5, wherein the bisphosphonic acid derivative is present as a monohydrate, dihydrate or trihydrate.

7. A formulation according to claim 5, wherein the bisphosphonic acid derivative is selected from the group consisting of alendronate sodium trihydrate, etidronate disodium and risedronate sodium monohydrate.

8. A formulation according to claim 7, wherein the bisphosphonic acid derivative is alendronate sodium trihydrate.

9. A formulation according to claim 7, wherein the bisphosphonic acid derivative is etidronate disodium.

10. A formulation according to claim 7, wherein the bisphosphonic acid derivative is risedronate sodium monohydrate.

11. A formulation according to claim 1, wherein the bisphosphonic acid derivative is present in the range of 0.5% to 40%.

12. A formulation according to claim 1, wherein the carbohydrate alcohol is selected from the group consisting of mannitol, maltitol, sorbitol, lactitol, erythritol and xylitol.

13. A formulation according to claim 12, wherein the carbohydrate alcohol is mannitol.

14. An oral formulation which includes an intragranular phase comprising a bisphosphonic acid derivative and a carbohydrate alcohol which is mannitol, together with an aqueous binder.

15. A formulation according to claim 14, which comprises 15 to 90% of the carbohydrate alcohol.

16. A formulation according to claim 15, which comprises 15 to 50% of the carbohydrate alcohol.

17. A formulation according to claim 16, which comprises 15 to 40% of the carbohydrate alcohol.

18. A formulation according to claim 1, wherein the intragranular phase further comprises one or more diluents and/or disintegrants.

19. A formulation according to claim 18, wherein the diluent is selected from the group consisting of microcrystalline cellulose, powdered cellulose, calcium phosphate-dibasic, calcium sulfate, dextrates, dextrins, alginates and dextrose excipients.

20. A formulation according to claim 19, wherein the diluent is microcrystalline cellulose.

21. A formulation according to claim 19, wherein the diluent is present in the range of 15 to 90%.

22. A formulation according to claim 18, wherein the disintegrant is selected from the group consisting of one or more of low substituted hydroxypropyl cellulose, carboxymethyl cellulose, calcium carboxymethylcellulose, sodium carboxymethyl cellulose, sodium starch glycollate, crospovidone, croscarmellose sodium, starch, crystalline cellulose, hydroxypropyl starch, and partially pregelatinized starch.

23. A formulation according to claim 22, wherein the disintegrant is sodium starch glycollate.

24. A formulation according to claim 22, wherein the disintegrant is present in the range of 5 to 20%.

25. A formulation according to claim 1, wherein the binder is selected from the group consisting of hydroxypropyl cellulose, hydroxypropyl methylcellulose, carboxymethyl cellulose sodium, polyvinylpyrrolidones, starches, gelatins and povidones.

26. A formulation according to claim 25, wherein the binder is starch.

27. A formulation according to claim 25, wherein the binder is present in the range of 1 to 15%.

28. A formulation according to claim 1, which further comprises one or more lubricants.

29. A formulation according to claim 28, wherein said lubricant is selected from the group consisting of talc, magnesium stearate, stearic acid, hydrogenated vegetable oils, glyceryl behenate, polyethylene glycols and derivatives thereof, sodium lauryl sulphate and sodium stearyl fumarate.

30. A formulation according to claim 29, wherein lubricant is magnesium stearate.

31. A formulation according to claim 28, wherein the lubricant is present in the range of 0.5 to 5%.

32. A formulation according to claim 1, which is a tablet.

33. A formulation according to claim 1, which is a capsule.

34. A formulation according to claim 1 that is substantially free of degradation products associated with the bisphosphonic acid derivative.

35. A formulation according to claim 1 prepared by a process comprising intimately mixing a bisphosphonic acid derivative and at least one carbohydrate alcohol to form a dry blend, wet granulating the dry blend with an aqueous binder so as to obtain an intragranular phase, and further formulating the resulting intragranular phase so as to provide the formulation.

36-52. (canceled)
